(19) United States
(12) Patent Application Publication
(10) Pub. No.: US 2024/0071619 A1
(43) Pub. Date: Feb. 29, 2024

(54) INFORMATION PROCESSING APPARATUS, INFORMATION PROCESSING METHOD, AND INFORMATION PROCESSING PROGRAM

(71) Applicant: FUJIFILM Corporation, Tokyo (JP)

(72) Inventors: Taiki FURUKAWA, Nagoya (JP); Shotaro MISAWA, Tokyo (JP); Ryuji KANO, Tokyo (JP); Hirokazu YARIMIZU, Tokyo (JP); Tomoki TANIGUCHI, Tokyo (JP); Tomoko OHKUMA, Tokyo (JP); Kohei ONODA, Tokyo (JP)

(73) Assignee: FUJIFILM Corporation, Tokyo (JP)

(21) Appl. No.: 18/457,331

(22) Filed: Aug. 28, 2023

(30) Foreign Application Priority Data
Aug. 31, 2022 (JP) 2022-138808

(51) Int. Cl. G16H 50/20; G16H 10/60

(52) U.S. Cl. CPC G16H 50/20; G16H 10/60

(57) ABSTRACT

An information processing apparatus including: plural models including at least a first model that uses first data as input to carry out a first prediction task and a second model that uses second data as input to carry out a second prediction task different from the first prediction task, and at least one processor, in which the processor is configured to: acquire information data representing information related to a specific target; select a selection model to be used for prediction from among the plural models based on the information data; and derive a degree of importance to the specific target for each item included in the information data by using the selection model.

### CROSS-REFERENCE TO RELATED APPLICATIONS

This application claims priority from Japanese Application No. 2022-138808, filed on Aug. 31, 2022, the entire disclosure of which is incorporated herein by reference.

### BACKGROUND

### Technical Field

The present disclosure relates to an information processing apparatus, an information processing method, and an information processing program.

### Related Art

There is known a technique of deriving a degree of importance to a specific target obtained by using a machine learning model for each item included in information data representing information related to the specific target. Examples of such a degree of importance include a degree of contribution of input data to derivation of output data in the machine learning model. Examples of the technique of deriving the degree of contribution include a method, such as local interpretable model-agnostic explanations (LIME).

For example, JP 2020-113218 A discloses a technique of deriving a degree of contribution for each word to a prediction model that uses word data related to a person as input and outputs an evaluation for the person.

By the way, in some cases, the machine learning model to be applied differs depending on a state of the specific target or the like. Therefore, in a case in which the degree of importance to the specific target is derived by applying an inappropriate machine learning model, the derived degree of importance is not appropriate in some cases.

### SUMMARY

The present disclosure has been made in view of the above circumstances, and is to provide an information processing apparatus, an information processing method, and an information processing program which can derive an appropriate degree of importance for each item included in information data representing information related to a specific target.

In order to achieve the above object, a first aspect of the present disclosure relates to an information processing apparatus comprising: a plurality of models including at least a first model that uses first data as input to carry out a first prediction task and a second model that uses second data as input to carry out a second prediction task different from the first prediction task; and at least one processor, in which the processor is configured to: acquire information data representing information related to a specific target; select a selection model to be used for prediction from among the plurality of models based on the information data; and derive a degree of importance to the specific target for each item included in the information data by using the selection model.

A second aspect relates to the information processing apparatus according to the first aspect, in which the processor is configured to: input the information data to the first model to acquire an output result related to the first prediction task; and select any one of the first model or the second model as the selection model based on the output result.

A third aspect relates to the information processing apparatus according to the first aspect, in which the first prediction task is a task for deriving a probability of a first state, the second prediction task is a task related to a second state different from the first state, and the processor is configured to: select the first model as the selection model in a case in which the probability of the first state exceeds a threshold value; and select the second model as the selection model in a case in which the probability of the first state is equal to or lower than the threshold value.

A fourth aspect relates to the information processing apparatus according to the first aspect, in which the information data is a document data group including a plurality of document data, the document data group is input to the first model to acquire an output result related to the first prediction task, any one of the first model or the second model is selected as the selection model based on the output result, and the degree of importance in the selection model is derived for each document data included in the document data group by using the selection model.

A fifth aspect relates to the information processing apparatus according to the first aspect, in which the first data is a plurality of first medical information data associated with each of a plurality of patients, and the specific target is a specific patient, the information data is a plurality of second medical information data associated with the specific patient, and the item is each of the plurality of second medical information data.

A sixth aspect relates to the information processing apparatus according to the first aspect, in which each of the first prediction task and the second prediction task is a medical-related task.

A seventh aspect relates to the information processing apparatus according to the sixth aspect, in which the first prediction task is a mortality prediction task, and the second prediction task is a long-term hospitalization prediction task or a complication prediction task.

In addition, in order to achieve the above object, an eighth aspect of the present disclosure relates to an information processing method executed by a processor of an information processing apparatus including a plurality of models including at least a first model that uses first data as input to carry out a first prediction task and a second model that uses second data as input to carry out a second prediction task different from the first prediction task, and at least one processor, the information processing method comprising: acquiring information data representing information related to a specific target; selecting a selection model to be used for prediction from among the plurality of models based on the information data; and deriving a degree of importance to the specific target for each item included in the information data by using the selection model.

In addition, in order to achieve the above object, a ninth aspect of the present disclosure relates to an information processing program causing a processor of an information processing apparatus including a plurality of models including at least a first model that uses first data as input to carry out a first prediction task and a second model that uses second data as input to carry out a second prediction task different from the first prediction task, and at least one processor, to execute a process comprising: acquiring information data representing information related to a specific target; selecting a selection model to be used for prediction from among the plurality of models based on the information data; and deriving a degree of importance to the specific target for each item included in the information data by using the selection model.

According to the present disclosure, an appropriate degree of importance can be derived for each item included in the information data representing the information related to the specific target.

### BRIEF DESCRIPTION OF THE DRAWINGS

FIG. 1 is a configuration diagram schematically showing one example of an overall configuration of an information processing system according to an embodiment.

FIG. 2 is a diagram for describing one example of patient information.

FIG. 3 is a block diagram showing one example of a configuration of an information processing apparatus according to a first embodiment.

FIG. 4 is a diagram for describing input and output of a mortality prediction model.

FIG. 5 is a diagram showing an outline of processing in a training phase of the mortality prediction model.

FIG. 6 is a diagram for describing input and output of a long-term hospitalization prediction model.

FIG. 7 is a diagram showing an outline of processing in a training phase of the long-term hospitalization prediction model.

FIG. 8 is a functional block diagram showing one example of a configuration of the information processing apparatus according to the first embodiment.

FIG. 9 is a diagram for describing an action of the information processing apparatus according to the first embodiment.

FIG. 10 is a diagram for describing an action of the information processing apparatus according to the first embodiment.

FIG. 11 is a flowchart showing one example of a flow of information processing by the information processing apparatus according to the first embodiment.

FIG. 12 is a diagram showing one example of a state in which document data is displayed on a display unit.

FIG. 13 is a block diagram showing one example of a configuration of a storage provided in an information processing apparatus according to a second embodiment.

FIG. 14 is a functional block diagram showing one example of a configuration of the information processing apparatus according to the second embodiment.

FIG. 15 is a diagram for describing an action of the information processing apparatus according to the second embodiment.

FIG. 16 is a diagram for describing an action of the information processing apparatus according to the second embodiment.

FIG. 17 is a diagram for describing a weight with respect to a mortality prediction evaluation value and a weight with respect to a long-term hospitalization prediction result.

FIG. 18 is a flowchart showing one example of a flow of information processing by the information processing apparatus according to the second embodiment.

FIG. 19 is a diagram for describing an action of the information processing apparatus according to the second embodiment.

### DETAILED DESCRIPTION

Hereinafter, the description of embodiments of the present disclosure will be made in detail with reference to the drawings. It should be noted that the present embodiment does not limit the technique of the present disclosure.

### First Embodiment

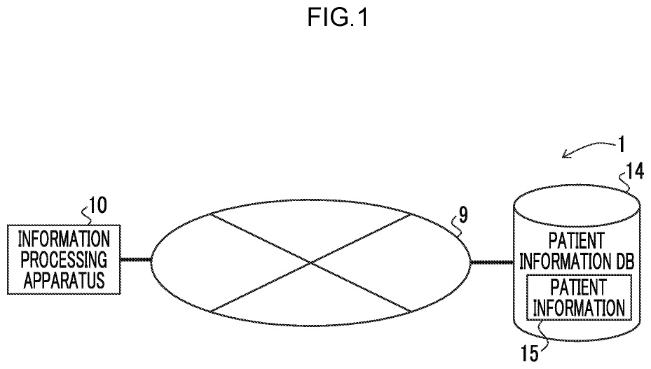

First, one example of an overall configuration of an information processing system according to the present embodiment will be described. FIG. 1 shows a configuration diagram showing one example of an overall configuration of an information processing system 1 according to the present embodiment. As shown in FIG. 1, the information processing system 1 according to the present embodiment comprises an information processing apparatus 10 and a patient information database (DB) 14. The information processing apparatus 10 and the patient information DB 14 are connected to each other via a network 9 by the wired communication or the wireless communication.

Patient information 15 related to a plurality of patients is stored in the patient information DB 14. The patient information DB 14 is realized by a storage medium, such as a hard disk drive (HDD), a solid state drive (SSD), and a flash memory, provided in a server apparatus in which a software program for providing functions of a database management system (DBMS) to a general-purpose computer is installed.

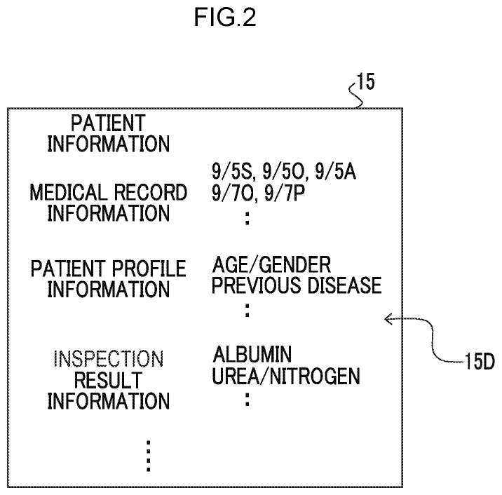

As one example, the patient information 15 according to the present embodiment is document data 15D representing a document related to medical care of a specific patient. As shown in FIG. 2, the document data 15D includes, for example, medical record information, patient profile information, and inspection result information. It should be noted that, in the present embodiment, the “document” is information in which at least one of a word or a sentence is a constituent element. For example, the document may include only one word, or may include a plurality of sentences. In the example shown in FIG. 2, as the document data 15D which is the medical record information, five of “9/5S”, “9/5O”, “9/5A”, “9/7O”, and “9/7P” are shown. In addition, as the document data 15D which is the patient profile information, two of “age/gender” and “past medical history” are shown. In addition, as the document data 15D which is the inspection result information, two of “albumin” (inspection value of albumin) and “urea/nitrogen” (inspection value of urea and inspection value of nitrogen) are shown.

The patient information 15 is stored in the patient information DB 14 in association with identification information for identifying the patient for each specific patient.

The specific patient according to the present embodiment is one example of a specific target according to the present disclosure, the patient information 15 according to the present embodiment is one example of information data according to the present disclosure, and the document data 15D according to the present embodiment is one example of an item according to the present disclosure.

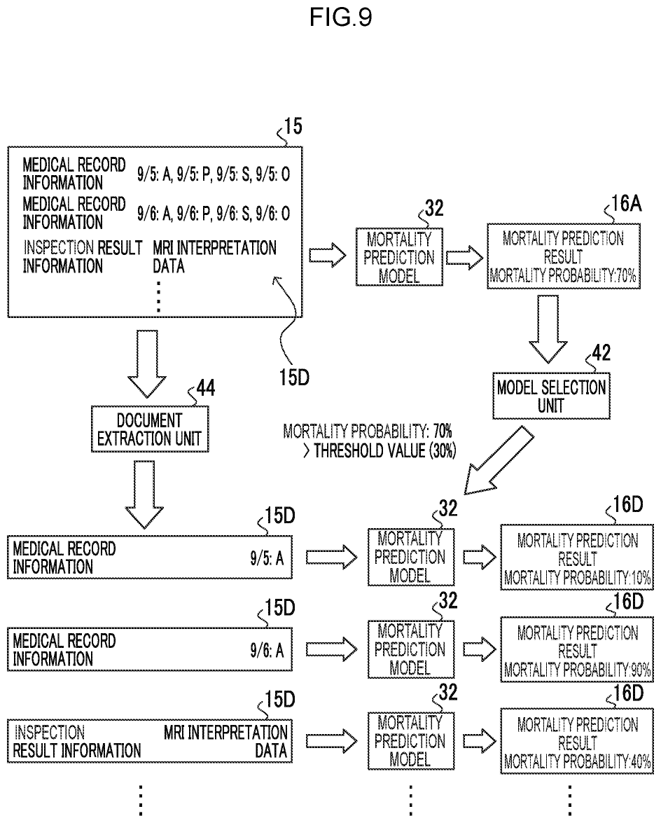

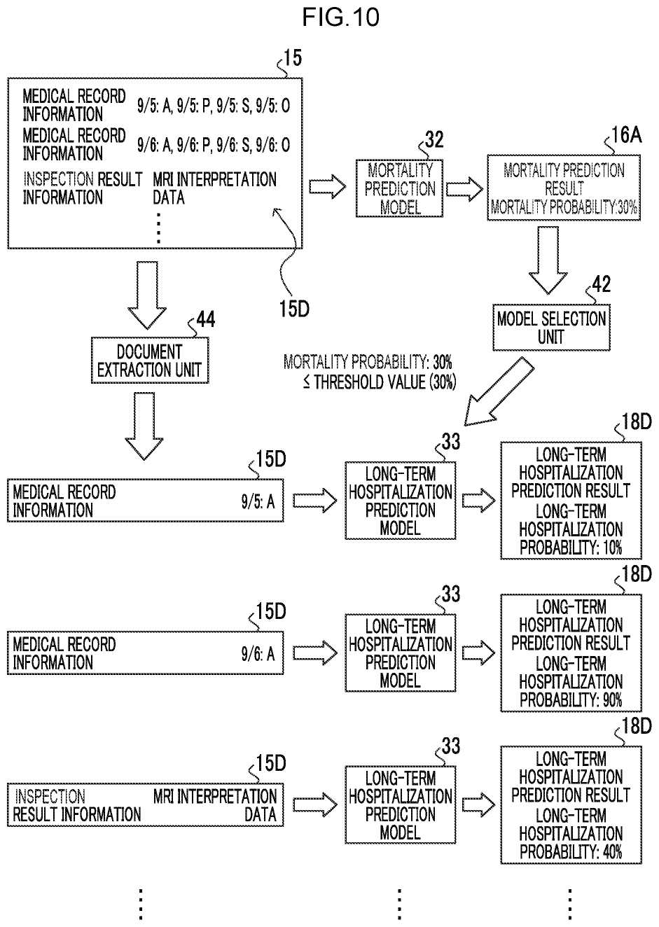

The information processing apparatus 10 is an apparatus that selects an appropriate model used to predict the medical care of the specific patient from among a mortality prediction model 32 and a long-term hospitalization prediction model 33, which are to be described below (see FIGS. 9 and 10), based on the patient information 15 of the specific patient, and provides information to a user, such as a doctor, by using the selected model.

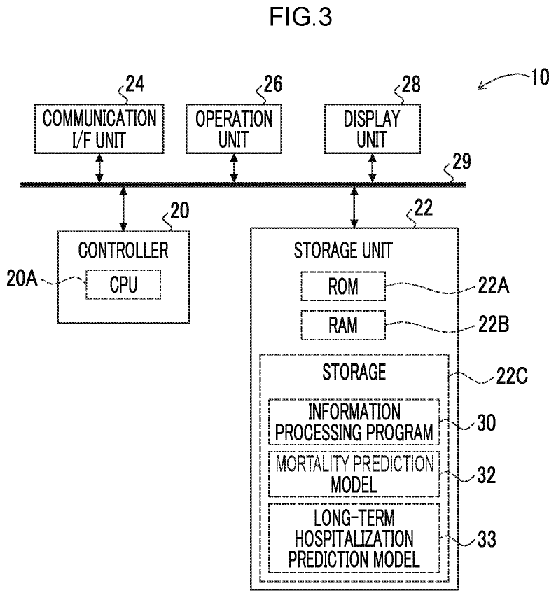

As shown in FIG. 3, the information processing apparatus 10 according to the present embodiment comprises a controller 20, a storage unit 22, a communication interface (I/F) unit 24, an operation unit 26, and a display unit 28. The controller 20, the storage unit 22, the communication OF unit 24, the operation unit 26, and the display unit 28 are connected to each other via a bus 29 such as a system bus or a control bus so that various types of information can be exchanged.

The controller 20 according to the present embodiment controls an overall operation of the information processing apparatus 10. The controller 20 is a processor, and comprises a central processing unit (CPU) 20A. In addition, the controller 20 is connected to the storage unit 22 to be described below. It should be noted that the controller 20 may comprise a graphics processing unit (GPU).

The operation unit 26 is used by the user to input, for example, an instruction or various types of information related to the prognosis prediction of the specific patient. The operation unit 26 is not particularly limited, and examples thereof include various switches, a touch panel, a touch pen, and a mouse. The display unit 28 displays a mortality prediction result 16, the document data 15D, various types of information, and the like. It should be noted that the operation unit 26 and the display unit 28 may be integrated into a touch panel display.

The communication OF unit 24 performs communication of various types of information with the patient information DB 14 via the network 9 by the wireless communication or the wired communication. The information processing apparatus 10 receives the patient information 15 from the patient information DB 14 via the communication OF unit 24 by the wireless communication or the wired communication.

The storage unit 22 comprises a read only memory (ROM) 22A, a random access memory (RAM) 22B, and a storage 22C. Various programs and the like executed by the CPU 20A are stored in the ROM 22A in advance. Various data are transitorily stored in the RAM 22B. The storage 22C stores an information processing program 30, the mortality prediction model 32, the long-term hospitalization prediction model 33, various types of other information, and the like executed by the CPU 20A. The storage 22C is a non-volatile storage unit, and is, for example, an HDD or an SSD.

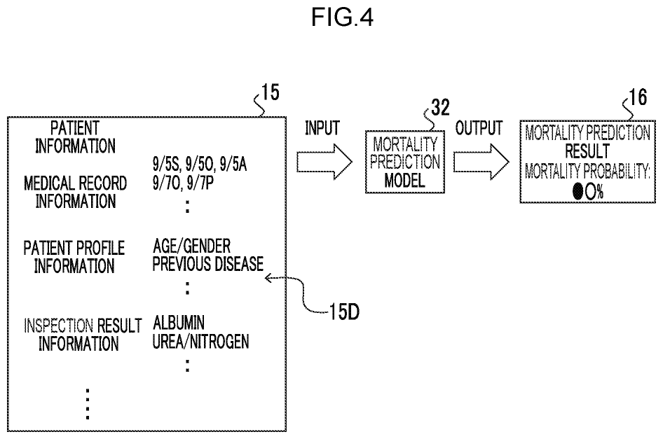

In the mortality prediction model 32 according to the present embodiment is a model that outputs a probability that the patient is in a death state, specifically, a mortality probability as a mortality prediction result 16 in a case in which the patient information 15 is input, as shown in FIG. 4. The death state according to the present embodiment is one example of a first state according to the present disclosure, and the mortality prediction model 32 according to the present embodiment is one example of a first model according to the present disclosure.

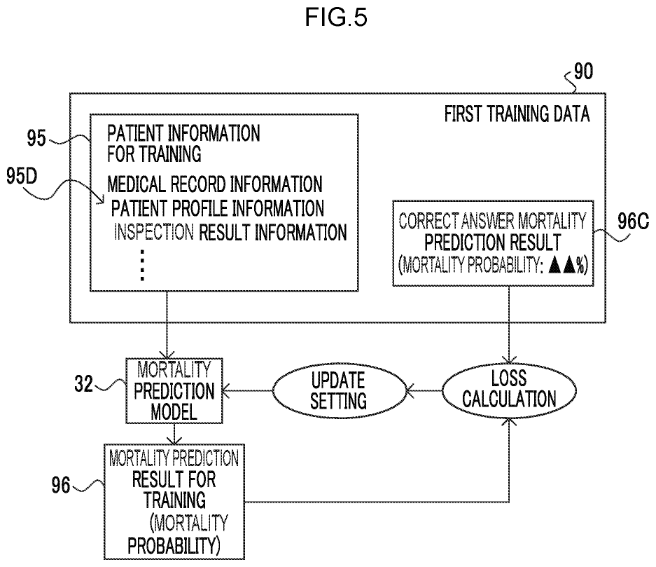

As shown in FIG. 5 as one example, the mortality prediction model 32 according to the present embodiment is trained by being given first training data 90, which is also called train data or teacher data, in a training phase. The first training data 90 is a set of patient information for training 95 and a correct answer mortality prediction result 96C. The first training data 90 includes a plurality of document data for training 95D related to the medical care of a certain patient. The correct answer mortality prediction result 96C is, for example, the mortality probability obtained from a result of actually observing the prognosis of the patient. Specifically, it is assumed that “the mortality probability of the patient who has actually died is 1 (100%)” and “the mortality probability of a patient who has not died is 0 (0%)”. It should be noted that the mortality probability is not limited to 100% and 0%, and various adjustments can be made. For example, in a case in which a period until death is long, the mortality probability may be reduced from 100%. It should be noted that, the present disclosure is not limited to the present embodiment, and as the correct answer mortality prediction result 96C, for example, the mortality probability actually given to the patient by a doctor with reference to the document data for training 95D may be used.

In the training phase, the patient information for training 95 is vectorized and input to the mortality prediction model 32 for each document data for training 95D. The mortality prediction model 32 outputs a mortality prediction result for training 96 to the patient information for training 95. A loss calculation of the mortality prediction model 32 using a loss function is performed based on the mortality prediction result for training 96 and the correct answer mortality prediction result 96C. Then, various coefficients of the mortality prediction model 32 are subjected to update setting according to a result of the loss calculation, and the mortality prediction model 32 is updated according to the update setting.

In the training phase, the series of pieces of processing of the input of the patient information for training 95 to the mortality prediction model 32, the output of the mortality prediction result for training 96 from the mortality prediction model 32, the loss calculation, the update setting, and the update of the mortality prediction model 32 are repeatedly performed while exchanging the first training data 90. The series of repetitions are terminated in a case in which the prediction accuracy of the mortality prediction result for training 96 with respect to the correct answer mortality prediction result 96C reaches a predetermined set level. As described above, the trained mortality prediction model 32 is generated.

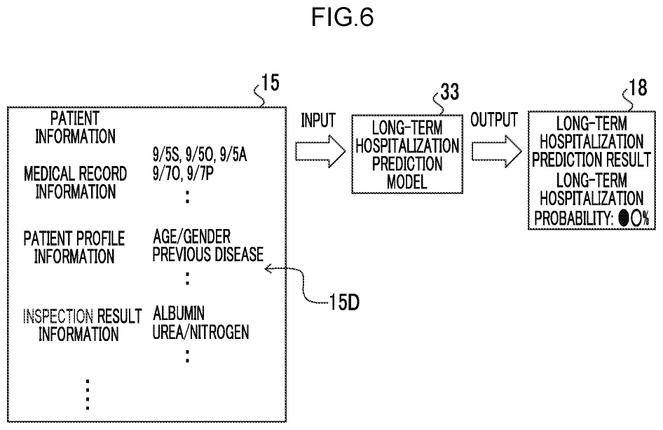

On the other hand, the long-term hospitalization prediction model 33 according to the present embodiment is a model that outputs a probability that the patient is in a long-term hospitalization state, specifically, a long-term hospitalization probability as a long-term hospitalization prediction result 18 in a case in which the patient information 15 is input, as shown in FIG. 6. The long-term hospitalization state according to the present embodiment is one example of a second state according to the present disclosure, and the long-term hospitalization prediction model 33 according to the present embodiment is one example of a second model according to the present disclosure.

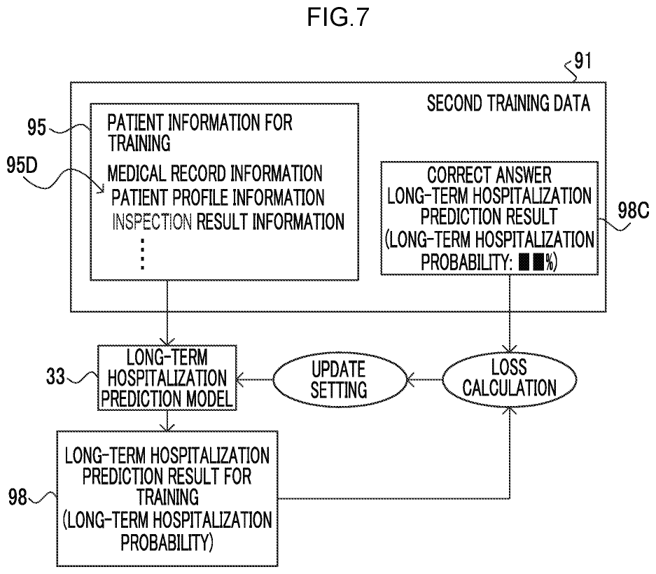

As shown in FIG. 7 as one example, the long-term hospitalization prediction model 33 according to the present embodiment is trained by being given second training data 91, which is also called train data or teacher data, in a training phase. The second training data 91 is a set of patient information for training 95 and a correct answer long-term hospitalization prediction result 98C. The second training data 91 includes a plurality of document data for training 95D related to the medical care of a certain patient. The correct answer long-term hospitalization prediction result 98C is the long-term hospitalization probability obtained from the actual number of the hospitalization days of the patient and the like by referring to the document data for training 95D by the doctor. It should be noted that the patient information for training 95 included in the first training data 90 used to train the mortality prediction model 32 and the patient information for training 95 included in the second training data 91 used to train the long-term hospitalization prediction model 33 may be the same as each other, may be different from each other, and may be partially the same as each other.

In the training phase, the patient information for training 95 is vectorized and input to the long-term hospitalization prediction model 33 for each document data for training 95D. The long-term hospitalization prediction model 33 outputs a long-term hospitalization prediction result for training 98 to the patient information for training 95. A loss calculation of the long-term hospitalization prediction model 33 using a loss function is performed based on the long-term hospitalization prediction result for training 98 and the correct answer long-term hospitalization prediction result 98C. Then, various coefficients of the long-term hospitalization prediction model 33 are subjected to update setting according to a result of the loss calculation, and the long-term hospitalization prediction model 33 is updated according to the update setting.

In the training phase, the series of pieces of processing of the input of the patient information for training 95 to the long-term hospitalization prediction model 33, the output of the long-term hospitalization prediction result for training 98 from the long-term hospitalization prediction model 33, the loss calculation, the update setting, and the update of the long-term hospitalization prediction model 33 are repeatedly performed while exchanging the second training data 91. The series of repetitions are terminated in a case in which the prediction accuracy of the long-term hospitalization prediction result for training 98 with respect to the correct answer long-term hospitalization prediction result 98C reaches a predetermined set level. As described above, the trained long-term hospitalization prediction model 33 is generated.

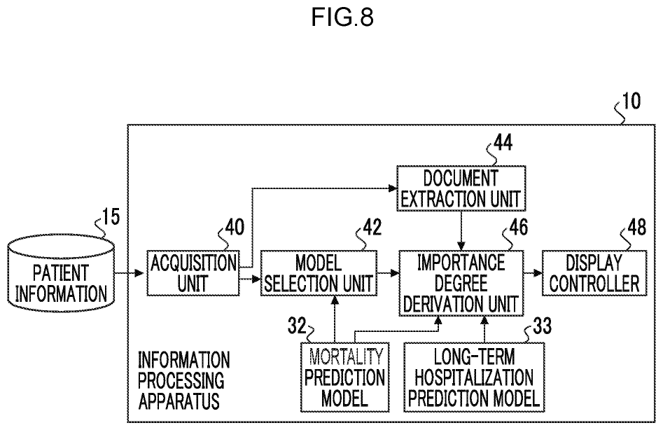

Further, FIG. 8 shows a functional block diagram of one example of the configuration of the information processing apparatus 10 according to the present embodiment. As shown in FIG. 8, the information processing apparatus 10 comprises an acquisition unit 40, a model selection unit 42, a document extraction unit 44, an importance degree derivation unit 46, and a display controller 48. As one example, in the information processing apparatus 10 according to the present embodiment, in a case in which the CPU 20A of the controller 20 executes the information processing program 30 stored in the storage 22C, the CPU 20A functions as the acquisition unit 40, the model selection unit 42, the document extraction unit 44, the importance degree derivation unit 46, and the display controller 48.

The acquisition unit 40 acquires the patient information 15 of the specific patient, which is a prediction target, from the patient information DB 14. As one example, in a case in which the acquisition unit 40 according to the present embodiment receives patient identification information representing the specific patient who is a target of the prognosis prediction, the acquisition unit 40 acquires the patient information 15 corresponding to the received patient identification information from the patient information DB 14 via the network 9. The acquisition unit 40 outputs the acquired patient information 15 to the model selection unit 42 and the document extraction unit 44.

The model selection unit 42 selects the selection model to be used for prediction from among the mortality prediction model 32 and the long-term hospitalization prediction model 33 based on the patient information 15. It should be noted that, here, the model selected by the model selection unit 42 from among the mortality prediction model 32 and the long-term hospitalization prediction model 33 is referred to as the “selection model”.

Specifically, as shown in FIGS. 9 and 10, as one example, the model selection unit 42 according to the present embodiment vectorizes all the document data 15D included in the patient information 15, inputs the vectorized document data 15D to the mortality prediction model 32, and acquires an output mortality prediction result 16A in a unit of the patient information. In other words, the model selection unit 42 derives the mortality prediction result 16A in a unit of the patient information by using the mortality prediction model 32. In a case in which the derived mortality prediction result 16A in a unit of the patient information exceeds a threshold value, the model selection unit 42 selects the mortality prediction model 32 as the selection model. In addition, in a case in which the mortality prediction result 16A in a unit of the patient information is equal to or lower than the threshold value, the model selection unit 42 selects the long-term hospitalization prediction model 33 as the selection model. It should be noted that, in the example shown in FIG. 9 and FIG. 10, the threshold value as a reference for selecting which of the mortality prediction model 32 and the long-term hospitalization prediction model 33 is selected is set to “30%”. However, the specific value of the threshold value is not particularly limited, and the value need only be a value for determining whether or not the mortality probability is high. In other words, based on the mortality prediction result 16A in a unit of the patient information, the model selection unit 42 selects the mortality prediction model 32 in a case in which it is predicted that the mortality probability is high, and selects the long-term hospitalization prediction model 33 in a case in which it is predicted that the mortality probability is not high.

The model selection unit 42 outputs information representing the selection model selected in this way to the importance degree derivation unit 46.

The document extraction unit 44 extracts the document data 15D from the patient information 15 based on a predetermined reference. As one example, the document extraction unit 44 according to the present embodiment extracts the document data 15D in a unit of a single sentence, by using one sentence included in the patient information 15 as one document data 15D. It should be noted that the reference for extracting the document data 15D from the patient information 15 is not particularly limited, and for example, the association date may be the same as the reference. In such a case, for example, in the example shown in FIGS. 9 and 10, as one document data 15D, “9/5: A, 9/5: P, 9/5: S, 9/5: O” is extracted. The document extraction unit 44 outputs the extracted document data 15D to the importance degree derivation unit 46.

The importance degree derivation unit 46 inputs each document data 15D to the selection model selected by the model selection unit 42 from among the mortality prediction model 32 and the long-term hospitalization prediction model 33, and acquires the output prediction result. Specifically, in a case in which the selection model is the mortality prediction model 32, as shown in FIG. 9, the importance degree derivation unit 46 vectorizes the document data 15D, inputs the vectorized document data 15D to the mortality prediction model 32, and acquires the output mortality prediction result 16D in a unit of the document. On the other hand, in a case in which the selection model is the long-term hospitalization prediction model 33, as shown in FIG. 10, the importance degree derivation unit 46 vectorizes the document data 15D, inputs the vectorized document data 15D to the long-term hospitalization prediction model 33, and acquires the output long-term hospitalization prediction result 18D in a unit of the document.

The importance degree derivation unit 46 derives the degree of importance to the specific patient based on the acquired mortality prediction result 16D in a unit of the document or the acquired long-term hospitalization prediction result 18D in a unit of the document. The degree of importance according to the present embodiment has a correlation with the output of the selection model in a case in which the document data 15D is input. In the present embodiment, the document data 15D is used as input, and a value obtained by converting the probability expressed by a percentage output from the selection model into a small number is adopted as the degree of importance. For example, the degree of importance is “0.9” in a case in which the mortality prediction result 16D in a unit of the document is 90%, the degree of importance is “0.3” in a case in which the mortality prediction result 16D in a unit of the document is 30%, and the degree of importance is “0.9” in a case in which the long-term hospitalization prediction result 18D in a unit of the document is 90%. Therefore, the degree of importance according to the present embodiment is a value equal to or larger than 0 and equal to or smaller than 1. It should be noted that, in the present embodiment, the degree of importance is represented as a specific numerical value, but may be represented by, for example, “high”, “medium”, “low”, or the like. The importance degree derivation unit 46 outputs the degree of importance derived for each document data 15D to the display controller 48.

The display controller 48 specifies the document data 15D, which is a display target, from among all the plurality of document data 15D included in the patient information 15 based on the degree of importance for each document data 15D. For example, the display controller 48 specifies a predetermined number of the document data 15D as the display targets in descending order of the degree of importance. In addition, the display controller 48 specifies the document data 15D of which the degree of importance is equal to or higher than a predetermined value as the display target. It should be noted that, in a case of specifying the display target, the display controller 48 may select the document data 15D one by one by using a method of Beam Search. In such a case, first, the display controller 48 extracts the highest K document data 15D in the ranking of the degree of importance from all the document data 15D included in the patient information 15 as the document data 15D to which a first display priority having the highest display priority is given. Then, other document data 15D are added to the extracted document data 15D and ranked based on the degree of importance, and a second display priority, which is the next to the first display priority, is given to the highest K document data 15D. This processing is repeated until a predetermined number of the document data 15D are specified or the total length obtained by adding the lengths of all the document data 15D to which the display priority is given reaches a predetermined length.

In addition, the display controller 48 specifies a display order in which the document data 15D is displayed based on the degree of importance. For example, the display controller 48 specifies the display order such that the display priority is raised in descending order of the degree of importance. It should be noted that the display controller 48 may adopt, as the display order, a time-series order based on the date and time associated with the document data 15D. In a case in which the display order is the time-series order, the display priority is higher as the date and time are newer. In addition, the display order in which the order according to the degree of importance and the time-series order are combined may be adopted. It should be noted that, in such a case, the burden on the user who reads the document data 15D is larger as the document data 15D is longer, and thus the length of the document data 15D may be added as a penalty. Specifically, the penalty that is larger as the length of the document data 15D is longer may be added.

It should be noted that, in a case in which at least one of the display target or the display order is determined in advance, the display controller 48 need only specify which of the display target and the display order is not determined in advance, and may omit the specification of the display target and the specification of the display order in a case in which both the display target and the display order are determined in advance. For example, in a case in which it is determined in advance that all the document data 15D are used as the display targets, the display controller 48 need only specify the display order. In addition, the display target may be specified by the method described above and the display order may be specified by another method. Alternatively, the display target may be specified by another method and only the display order may be specified by the method described above. For example, in a case in which there are an electronic medical record, a radiological inspection report, a nursing record, and the like as medical documents, the display target may be limited to the electronic medical record, all of the electronic medical records may be displayed, the electronic medical records may be rearranged in the specified display order. In addition, an embodiment may be adopted in which, as a method of specifying the display target, the document data 15D, which is specified as the display target, is displayed with more emphasis than the document data 15D, which is not the display target. For example, the document data 15D, which is specified as the display target, may be highlighted and displayed, and the document, which is not the display target, may be grayed out.

In addition, the display controller 48 performs control of displaying the document data 15D specified as the display target on the display unit 28 in the specified display order.

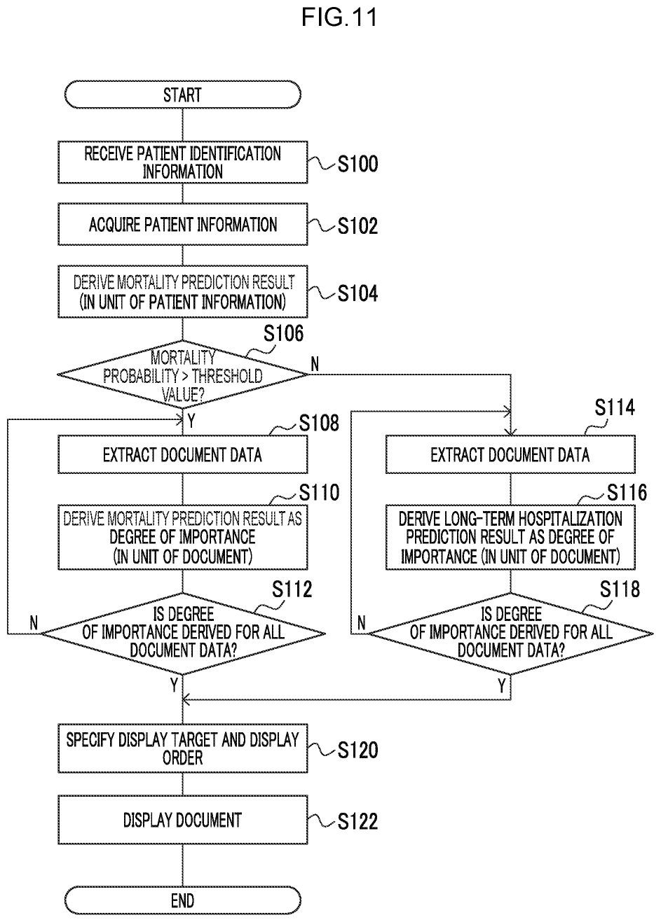

Hereinafter, an action of the information processing apparatus 10 according to the present embodiment will be described with reference to the drawings. FIG. 11 shows a flowchart showing one example of a flow of information processing executed by the information processing apparatus 10 according to the present embodiment. The information processing apparatus 10 according to the present embodiment executes the information processing shown in FIG. 11 in a case in which the CPU 20A of the controller 20 executes the information processing program 30 stored in the storage 22C based on a start instruction or the like of the user performed by the operation unit 26, as one example.

In step S100 of FIG. 11, as described above, the acquisition unit 40 receives the patient identification information designated by the user using the operation unit 26. In next step S102, as described above, the acquisition unit 40 acquires the patient information 15 associated with the patient identification information from the patient information DB 14 via the network 9.

In next step S104, as described above, the model selection unit 42 inputs all the document data 15D included in the patient information 15 to the mortality prediction model 32, and derives the mortality prediction result 16A in a unit of the patient information.

In next step S106, as described above, the model selection unit 42 determines whether or not the mortality probability, which is the mortality prediction result 16A in a unit of the patient information, exceeds the threshold value. As shown in FIG. 9, in a case in which the mortality probability exceeds the threshold value, a positive determination is made in the determination in step S106, and the processing proceeds to step S108. In such a case, the model selection unit 42 selects the mortality prediction model 32 as the selection model.

In step S108, the document extraction unit 44 extracts one document data 15D from the patient information 15 acquired in step S102, as described above. In next step S110, as described above, the importance degree derivation unit 46 derives the mortality prediction result 16D in a unit of the document as the degree of importance. Specifically, the importance degree derivation unit 46 inputs the document data 15D extracted in step S108 to the mortality prediction model 32, and acquires the output mortality prediction result 16D in a unit of the document as the degree of importance.

In next step S112, the document extraction unit 44 determines whether or not the mortality prediction is performed for all the document data 15D included in the patient information 15. In a case in which the mortality prediction is not yet performed for all the document data 15D, a negative determination is made in the determination in step S112, the processing returns to step S108, and the pieces of processing of steps S108 and S110 are repeated. On the other hand, in a case in which the mortality prediction is performed for all the document data 15D, a positive determination is made in the processing of step S112, and the processing proceeds to step S120.

On the other hand, in step S106, as shown in FIG. 10, in a case in which the mortality probability of the mortality prediction result 16A in a unit of the patient information is equal to or lower than the threshold value, a negative determination is made in the determination in step S106, and the processing proceeds to step S114. In such a case, the model selection unit 42 selects the long-term hospitalization prediction model 33 as the selection model.

In step S114, as described above, the document extraction unit 44 derives the long-term hospitalization prediction result 18D in a unit of the document as the degree of importance. Specifically, the importance degree derivation unit 46 inputs the document data 15D extracted in step S114 to the long-term hospitalization prediction model 33, and acquires the output long-term hospitalization prediction result 18D in a unit of the document as the degree of importance.

In next step S118, the document extraction unit 44 determines whether or not the long-term hospitalization prediction is performed for all the document data 15D included in the patient information 15. In a case in which the long-term hospitalization prediction is not yet performed for all the document data 15D, a negative determination is made in the determination in step S118, the processing returns to step S114, and the pieces of processing of steps S114 and S116 are repeated. On the other hand, in a case in which the long-term hospitalization prediction is performed for all the document data 15D, a positive determination is made in the processing of step S118, and the processing proceeds to step S120.

In step S120, as described above, the display controller 48 specifies the display target from among all the document data 15D included in the patient information 15, and also specifies the display order of the document data 15D, which is the display target.

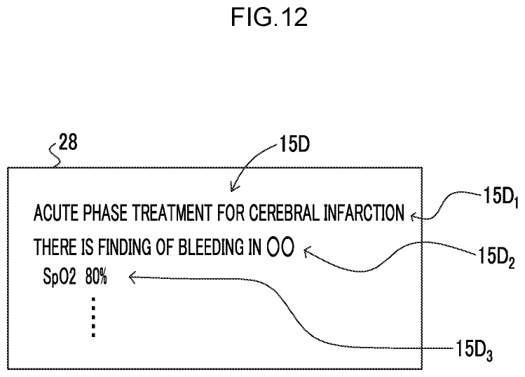

In next step S122, as described above, the display controller 48 displays the document corresponding to the document data 15D, which is the display target, on the display unit 28 in the specified display order. FIG. 12 is a diagram showing one example of a state in which the document data 15D, which is the display target, is displayed on the display unit 28 in the specified display order. In document data 15D₁ to 15D₃ shown in FIG. 12, the document data 15D₁ has the highest degree of importance and the document data 15D₃ has the lowest degree of importance. In this way, by displaying the document data 15D which is the display target specified in step S120 on the display unit 28 in the specified display order, useful information for the medical care of the specific patient is provided to the user in descending order of the degree of importance. In a case in which the processing of step S122 is terminated, the information processing shown in FIG. 11 is terminated.

As described above, the information processing apparatus 10 according to the present embodiment comprises the mortality prediction model 32 that uses the patient information 15 as input to carry out the mortality prediction task, and the long-term hospitalization prediction model 33 that uses the patient information 15 as input to carry out the long-term hospitalization prediction task.

The acquisition unit 40 acquires the patient information 15 representing the information related to the specific patient, and the model selection unit 42 selects the selection model to be used for prediction from among the mortality prediction model 32 and the long-term hospitalization prediction model 33 based on the mortality prediction result 16A in a unit of the patient information output from the mortality prediction model 32. In addition, the importance degree derivation unit 46 derives the degree of importance according to the mortality prediction result 16D in a unit of the document or the long-term hospitalization prediction result 18D in a unit of the document, which is output from the selection model.

As a result, with the information processing apparatus 10 according to the present embodiment, it is possible to derive an appropriate degree of importance to the specific patient for each document data 15D included in the patient information 15 representing the information related to the specific patient.

It should be noted that, in the present embodiment, the embodiment is described in which the mortality prediction model 32 that performs the mortality prediction and the long-term hospitalization prediction model 33 that performs the long-term hospitalization prediction are used, but the present disclosure is not limited to the present embodiment. For example, a complication prediction model that performs complication prediction may be used instead of the long-term hospitalization prediction model 33 or together with the long-term hospitalization prediction model 33. In a case in which three machine learning models of the mortality prediction model 32, the long-term hospitalization prediction model 33, and the complication prediction model are used, two threshold values described above need only be provided.

### Second Embodiment

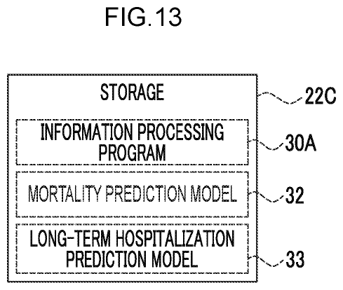

The information processing apparatus 10 according to the present embodiment will be described with reference to FIGS. 13 to 19. It should be noted that, since the information processing apparatus 10 according to the present embodiment has the same configuration as the information processing apparatus 10 according to the first embodiment, the same configuration will be described to that effect, and the detailed description thereof will be omitted.

First, the configuration of the information processing apparatus 10 according to the present embodiment will be described. As shown in FIG. 13, in the information processing apparatus 10 according to the present embodiment, an information processing program 30A is stored in the storage 22C instead of the information processing program 30 (see FIG. 3) according to the first embodiment.

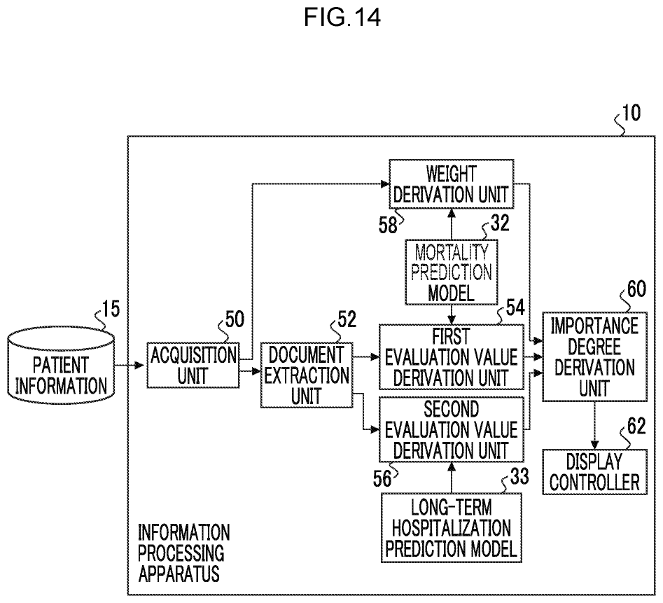

Further, FIG. 14 shows a functional block diagram of one example of the configuration of the information processing apparatus 10 according to the present embodiment. As shown in FIG. 14, the information processing apparatus 10 comprises an acquisition unit 50, a document extraction unit 52, a first evaluation value derivation unit 54, a second evaluation value derivation unit 56, a weight derivation unit 58, an importance degree derivation unit 60, and a display controller 62. As one example, in the information processing apparatus 10 according to the present embodiment, in a case in which the CPU 20A of the controller 20 executes the information processing program 30A stored in the storage 22C, the CPU 20A functions as the acquisition unit 50, the document extraction unit 52, the first evaluation value derivation unit 54, the second evaluation value derivation unit 56, the weight derivation unit 58, the importance degree derivation unit 60, and the display controller 62.

Similar to the acquisition unit 40 according to the first embodiment, the acquisition unit 50 acquires the patient information 15 of the specific patient, which is the prediction target, from the patient information DB 14. The acquisition unit 50 outputs the acquired patient information 15 to the document extraction unit 52 and the weight derivation unit 58.

Similar to the document extraction unit 44 according to the first embodiment, the document extraction unit 52 extracts the document data 15D from the patient information 15 based on the predetermined reference. The document extraction unit 52 outputs the extracted document data 15D to the first evaluation value derivation unit 54 and the second evaluation value derivation unit 56.

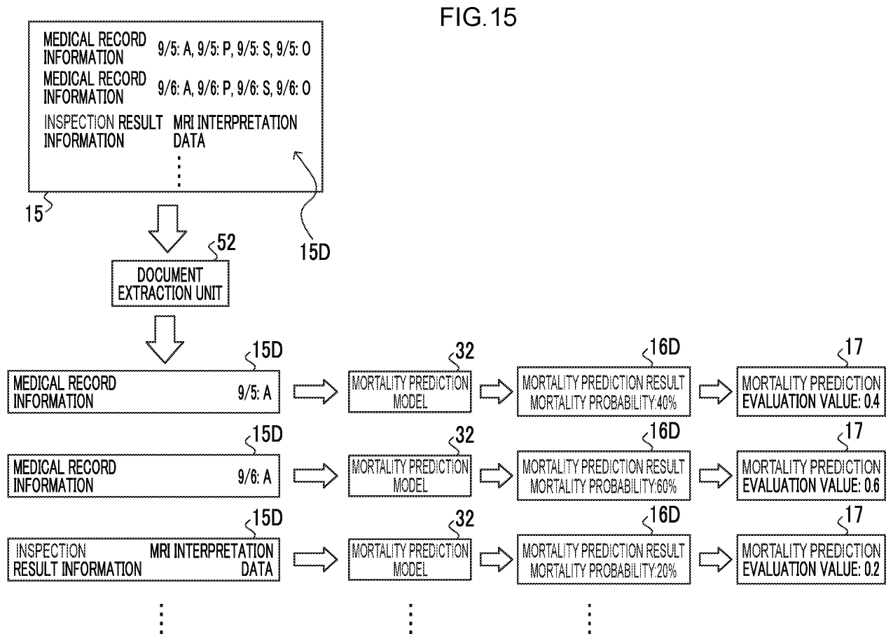

As shown in FIG. 15, the first evaluation value derivation unit 54 uses each document data 15D included in the patient information 15 as input of the mortality prediction model 32, and derives a mortality prediction evaluation value with respect to the mortality prediction result 16D in a unit of the document related to the mortality prediction.

As shown in FIG. 15, the first evaluation value derivation unit 54 vectorizes the document data 15D, inputs the vectorized document data 15D to the mortality prediction model 32, and acquires the output mortality prediction result 16D in a unit of the document. In addition, the first evaluation value derivation unit 54 derives the mortality prediction evaluation value 17 for evaluating the mortality prediction result 16D in a unit of the document. As one example, the mortality prediction evaluation value 17 according to the present embodiment adopts a value obtained by converting the mortality probability expressed by a percentage, which is the mortality prediction result 16D in a unit of the document, into a small number as the degree of importance. It should be noted that the mortality prediction evaluation value 17 is not limited to the present embodiment, and need only be an evaluation value that correlates with the mortality prediction result 16D in a unit of the document. The first evaluation value derivation unit 54 outputs the derived mortality prediction evaluation value 17 to the importance degree derivation unit 60.

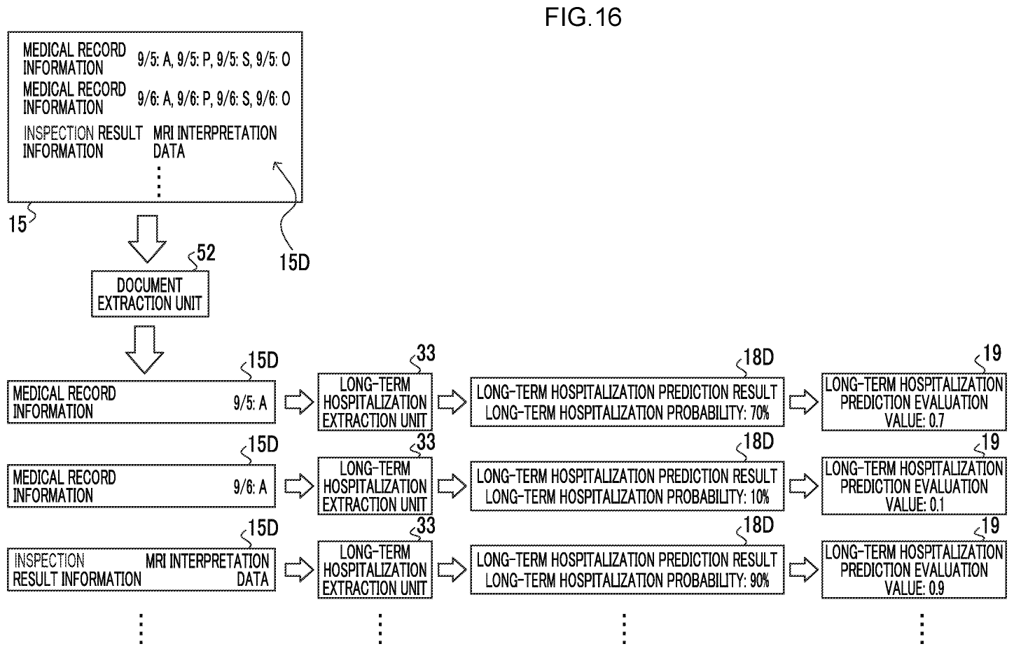

As shown in FIG. 16, the second evaluation value derivation unit 56 vectorizes the document data 15D, inputs the vectorized document data 15D to the long-term hospitalization prediction model 33, and acquires the output long-term hospitalization prediction result 18D in a unit of the document. In addition, the first evaluation value derivation unit 54 derives the long-term hospitalization prediction evaluation value 19 for evaluating the long-term hospitalization prediction result 18D in a unit of the document. As one example, the long-term hospitalization prediction evaluation value 19 according to the present embodiment adopts a value obtained by converting the long-term hospitalization probability expressed by a percentage, which is the long-term hospitalization prediction result 18D in a unit of the document, into a small number as the degree of importance. It should be noted that the long-term hospitalization prediction evaluation value 19 is not limited to the present embodiment, and need only be an evaluation value that correlates with the long-term hospitalization prediction result 18D in a unit of the document. The second evaluation value derivation unit 56 outputs the derived long-term hospitalization prediction evaluation value 19 to the importance degree derivation unit 60.

The weight derivation unit 58 derives a weight to be used for weighting with respect to each of the mortality prediction evaluation value 17 and the long-term hospitalization prediction evaluation value 19 based on the mortality prediction result 16A in a unit of the patient information. The weight derivation unit 58 outputs the derived weight to the importance degree derivation unit 60.

The importance degree derivation unit 60 derives the degree of importance to the specific patient for each document data 15D from the mortality prediction evaluation value 17 and the long-term hospitalization prediction evaluation value 19 weighted by the weight derived by the weight derivation unit 58.

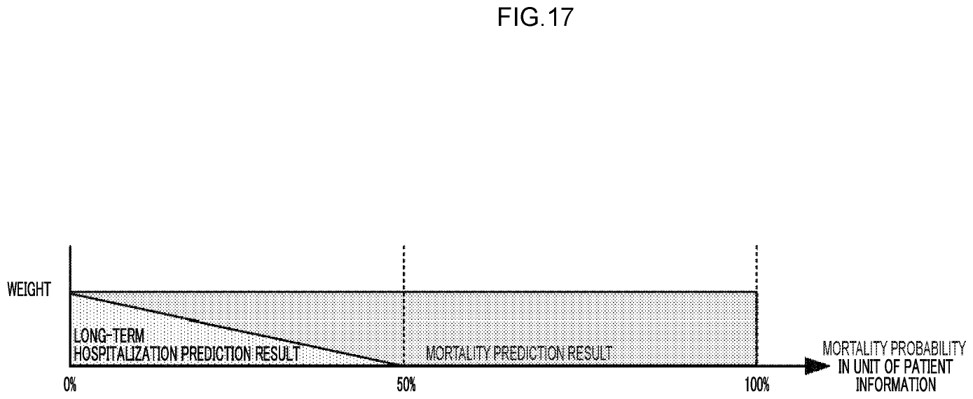

Here, the “weight” derived by the weight derivation unit 58 and the degree of importance derived by the importance degree derivation unit 60 according to the present embodiment will be described with reference to FIG. 17. As one example, in the present embodiment, it is more important that the patient is in the death state than in the long-term hospitalization state. Therefore, as shown in FIG. 17, the “weight” is determined in advance with reference to the mortality prediction result 16A in a unit of the patient information. As shown in FIG. 17, as the mortality probability, which is the mortality prediction result 16A in a unit of the patient information, is higher, the weight with respect to the mortality prediction evaluation value 17 (mortality prediction) is larger. In addition, as the mortality probability, which is the mortality prediction result 16A in a unit of the patient information, is higher, the weight with respect to the long-term hospitalization prediction evaluation value 19 (long-term hospitalization prediction) is larger.

The importance degree derivation unit 60 derives the degree of importance based on the following expression (1).

Degree of importance=(min(1,γ×mortality probability in unit of patient information)×mortality prediction evaluation value)+λ×(1−min value)×long-term hospitalization prediction evaluation value (1)

In the expression (1) described above, “min (1, γ×mortality probability in unit of patient information)” is the weight with respect to the mortality prediction evaluation value 17, and “λ×(1−min value)” is the weight with respect to the long-term hospitalization prediction evaluation value 19.

It should be noted that, “mortality probability in unit of patient information” in the expression (1) described above is a value obtained by converting the mortality probability expressed by a percentage, which is the mortality prediction result 16A in a unit of the patient information, into a small number.

In addition, “γ” and “λ” in the expression (1) described above are hyper parameters. γ is a parameter that represents a degree to which the mortality prediction result is considered to be important, and controls an inclination of a boundary line that is a boundary between the “long-term hospitalization prediction result” and the “mortality prediction result” in FIG. 17. As the γ is larger, a proportion of the “mortality prediction result” in FIG. 17 is higher, and the degree to which the mortality prediction result (mortality prediction evaluation value 17) is considered to be important is higher. FIG. 17 shows a case in which γ=2. In a case in which γ=2, in a case in which the mortality prediction result 16A in a unit of the patient information is equal to or higher than 50%, the weight with respect to the long-term hospitalization prediction evaluation value 19 is “0”, and the long-term hospitalization prediction evaluation value 19 is not used for derivation of the degree of importance.

On the other hand, λ is a coefficient for balancing the mortality prediction result and the long-term hospitalization prediction result. For example, the mortality probability, which is the mortality prediction result 16D in a unit of the document, is often a value in a range of 0% to 30%, and the long-term hospitalization probability, which is the long-term hospitalization prediction result 18D in a unit of the document, is often a value in a range of 20% to 100%. In the expression (1) described above, in a case in which λ is not used, the long-term hospitalization probability often shows a larger value than the mortality probability, and thus the long-term hospitalization prediction result (long-term hospitalization prediction evaluation value 19) is considered to be important. Therefore, by using λ as the hyper parameter, the mortality prediction result (mortality prediction evaluation value 17) and the long-term hospitalization prediction result (long-term hospitalization prediction evaluation value 19) are balanced. For example, λ may be determined manually. Further, for example, λ may be a value obtained by dividing a statistical value of the mortality prediction evaluation value 17 by an average value of the long-term hospitalization prediction evaluation value 19. It should be noted that examples of the statistical value in such a case include an average value and a median value.

Similar to the display controller 48 according to the first embodiment, the display controller 62 specifies the document data 15D, which is the display target, from among all the plurality of document data 15D included in the patient information 15 based on the degree of importance for each document data 15D. In addition, the display controller 62 specifies the display order in which the document data 15D is displayed based on the degree of importance. In addition, the display controller 62 performs control of displaying the document data 15D specified as the display target on the display unit 28 in the specified display order.

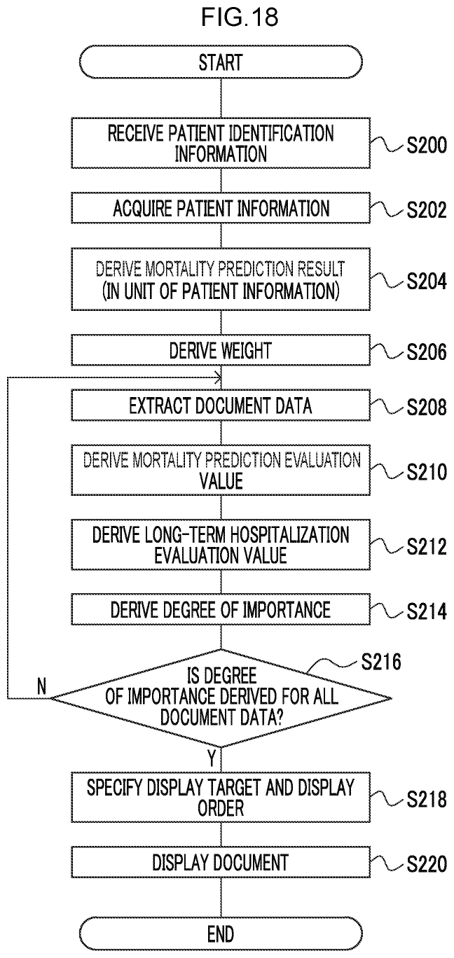

Hereinafter, an action of the information processing apparatus 10 according to the present embodiment will be described with reference to the drawings. FIG. 18 shows a flowchart showing one example of a flow of information processing executed by the information processing apparatus 10 according to the present embodiment. The information processing apparatus 10 according to the present embodiment executes the information processing shown in FIG. 18 in a case in which the CPU 20A of the controller 20 executes the information processing program 30A stored in the storage 22C based on a start instruction or the like of the user performed by the operation unit 26, as one example.

In step S200 of FIG. 18, the acquisition unit 50 receives the patient identification information designated by the user using the operation unit 26 in the same manner as in step S100 (see FIG. 11) of the information processing according to the first embodiment. In next step S202, the acquisition unit 50 acquires the patient information 15 associated with the patient identification information from the patient information DB 14 via the network 9 in the same manner as in the information processing step S102 (see FIG. 11) according to the first embodiment.

In next step S204, the weight derivation unit 58 inputs all the document data 15D included in the patient information 15 to the mortality prediction model 32, and derives the mortality prediction result 16A in a unit of the patient information in the same manner as in the information processing step S104 (see FIG. 11) according to the first embodiment.

In next step S206, as described above, the weight derivation unit 58 derives the weight to be used for weighting with respect to each of the mortality prediction evaluation value 17 and the long-term hospitalization prediction evaluation value 19 based on the mortality prediction result 16A in a unit of the patient information. Specifically, the weight derivation unit 58 according to the present embodiment derives the weight “min (1, γ×mortality probability in unit of patient information)” with respect to the mortality prediction evaluation value 17, and the weight “λ×(1−min value)” with respect to the long-term hospitalization prediction evaluation value 19.

In next step S208, the document extraction unit 52 extracts one document data 15D from the patient information 15 acquired in step S202, in the same manner as step S108 or S114 (see FIG. 11) of the information processing according to the first embodiment.

In next step S210, the first evaluation value derivation unit 54 inputs the document data 15D to the mortality prediction model 32, and derives the mortality prediction evaluation value 17 based on the output mortality prediction result 16D in a unit of the document.

In next step S212, the second evaluation value derivation unit 56 inputs the document data 15D to the long-term hospitalization prediction model 33, and derives the long-term hospitalization prediction evaluation value 19 based on the output long-term hospitalization prediction result 18D in a unit of the document.

In next step S214, as described above, the importance degree derivation unit 60 derives the degree of importance to specific patient for each document data 15D. Specifically, the importance degree derivation unit 60 derives the degree of importance to the specific patient for each document data 15D by the expression (1) described above by using the weight derived in step S206, the mortality prediction evaluation value 17 derived in step S210, and the long-term hospitalization prediction evaluation value 19 derived in step S212.

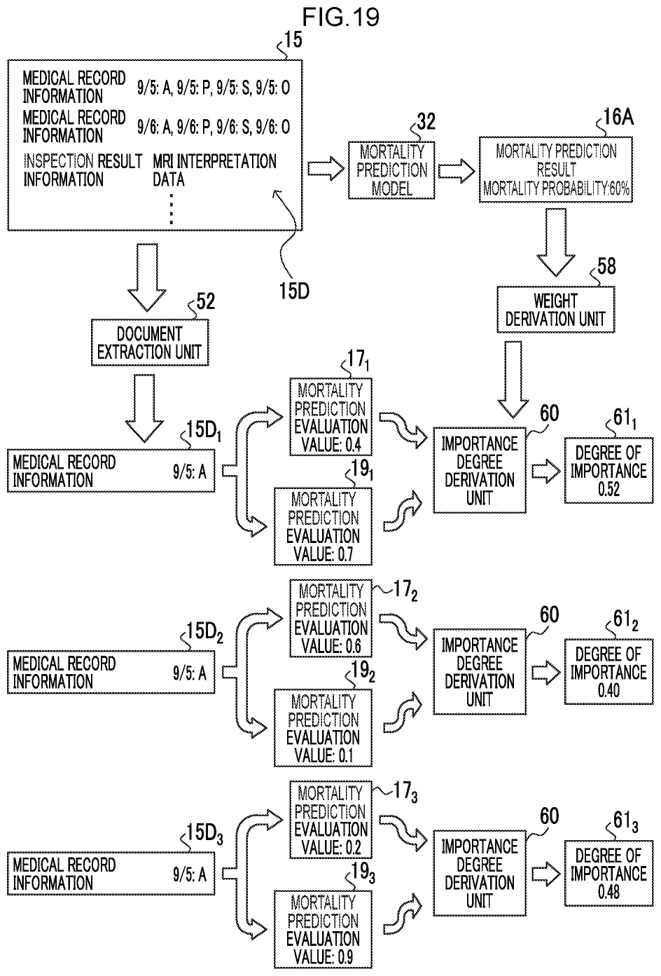

In FIG. 19, degrees of importance 61₁ to 61₃ of the document data 15D₁ to the document data 15D₃ in a case in which the hyper parameters γ and λ are both 1 and the mortality probability of the mortality prediction result 16A is 60% are shown. In such a case, the weight with respect to the mortality prediction evaluation value 17 is “min (1, 1×0.6)=0.6”. In addition, the weight with respect to the long-term hospitalization prediction evaluation value 19 is “1×(1−0.6)=0.4”.

In the example shown in FIG. 19, in a case of the document data 15D₁, since a mortality prediction evaluation value 17₁ is 0.4 and a long-term hospitalization prediction evaluation value 19₁ is 0.7, the degree of importance 61₁ is 0.6.×0.4+0.4×0.7=0.52. In addition, in a case of the document data 15D₂, since a mortality prediction evaluation value 17₂ is 0.6 and a long-term hospitalization prediction evaluation value 19₂ is 0.1, the degree of importance 61₂ is 0.6×0.6+0.4×0.1=0.40. In addition, in a case of the document data 15D₃, since a mortality prediction evaluation value 17₃ is 0.2 and a long-term hospitalization prediction evaluation value 19₃ is 0.9, the degree of importance 61₃ is 0.6×0.2+0.4×0.9=0.48.

In next step S216, the importance degree derivation unit 60 determines whether or not the degree of importance to the specific patient is derived for all the document data 15D included in the patient information 15. In a case in which the degree of importance to the specific patient is not yet derived for all the document data 15D, a negative determination is made in the determination in step S216, the processing returns to step S208, and the pieces of processing of steps S208 to S214 are repeated. On the other hand, in a case in which the degree of importance to the specific patient is derived for all the document data 15D, a positive determination is made in the determination in step S216, and the processing proceeds to step S218.

In step S218, as described above, the display controller 62 specifies the display target from among all the document data 15D included in the patient information 15, and also specifies the display order of the document data 15D, which is the display target. For example, in a case in which the display order is in descending order of the degree of importance 61, in the example shown in FIG. 19, the display controller 62 specifies the display order of the document data 15D₁ to the first order, which is highest, the display order of the document data 15D₃ to the second order, and the display order of the document data 15D₂ to the third order.

In next step S220, as described above, the display controller 62 displays the document corresponding to the document data 15D, which is the display target, on the display unit 28 in the specified display order. In this way, by displaying the document data 15D which is the display target specified in step S218 on the display unit 28 in the specified display order, useful information for the medical care of the specific patient is provided to the user in descending order of the degree of importance. In a case in which the processing of step S220 is terminated, the information processing shown in FIG. 18 is terminated.

As described above, the information processing apparatus 10 according to the present embodiment comprises the mortality prediction model 32 that uses the patient information 15 as input to carry out the mortality prediction task, and the long-term hospitalization prediction model 33 that uses the patient information 15 as input to carry out the long-term hospitalization prediction task. The acquisition unit 50 acquires the patient information 15 representing the information related to the specific patient. The first evaluation value derivation unit 54 uses each document data 15D included in the patient information 15 as input of the mortality prediction model 32, and derives the mortality prediction evaluation value 17 with respect to the mortality prediction result 16D in a unit of the document. The second evaluation value derivation unit 56 uses each document data 15D included in the patient information 15 as input of the long-term hospitalization prediction model 33, and derives the long-term hospitalization prediction evaluation value 19 with respect to the long-term hospitalization prediction result 18D in a unit of the document. The weight derivation unit 58 uses the patient information 15 as input of the mortality prediction model 32, acquires the mortality prediction result 16A in a unit of the patient information, and performs weighting with respect to each of the mortality prediction evaluation value 17 and the long-term hospitalization prediction evaluation value 19 by using the weight according to the mortality prediction result 16A in a unit of the patient information.

As a result, with the information processing apparatus 10 according to the present embodiment, it is possible to derive an appropriate degree of importance to the specific patient for each document data 15D included in the patient information 15 representing the information related to the specific patient.

It should be noted that, in the present embodiment, the embodiment is described in which the importance degree derivation unit 60 derives the degree of importance for each document data 15D, but an embodiment may be adopted in which at least one of the document data 15D related to the mortality prediction, which is the display target, or the display order is specified based on the weight derived by the weight derivation unit 58 without deriving the degree of importance. In addition, an embodiment may be adopted in which, instead of deriving the degree of importance, at least one of the document data 15D related to the long-term hospitalization prediction, which is the display target, or the display order is specified according to the weight derived by the weight derivation unit 58.

It should be noted that, in the first and second embodiments, the specific patient is adopted as one example of the specific target according to the present disclosure, but the specific target is not limited to this. For example, a specific company, a specific pet, a specific product, and the like may be used. In addition, in each of the each embodiment described above, the embodiment is described in which the first prediction task according to the present disclosure is the mortality prediction task in a task for generating a medical care summary related to the medical care of the specific patient, the first model according to the present disclosure is the mortality prediction model 32, the second prediction task according to the present disclosure is the long-term hospitalization prediction task, and the second model according to the present disclosure is the long-term hospitalization prediction model 33, but the present disclosure is not limited to this.

For example, the first prediction task according to the present disclosure may be a task for outputting an adoption probability that a document in the task for generating a summary of an in-office document in the specific company is adopted as a document for officers, and the first model according to the present disclosure may be a document-for-officer adoption model that outputs the adoption probability. In such a case, the second prediction task according to the present disclosure may be a task for outputting the adoption probability that the document is adopted as a document for a department, and the second model according to the present disclosure may be a department document adoption model that outputs the adoption probability. Further, in such a case, the degree of importance to the specific company need only be derived.

It should be noted that, in the first prediction task (first model) and the second prediction task (second model) according to the present disclosure, the first prediction task is a task that is more important to the specific target than the second prediction task. In the first and second embodiments, since “death” is more important than “long-term hospitalization”, the mortality prediction task is one example of a first prediction task according to the present disclosure, and the long-term hospitalization prediction task is one example of a second prediction task according to the present disclosure.

In addition, in the first and second embodiments, the embodiment is described in which two prediction models, the mortality prediction model 32 and the long-term hospitalization prediction model 33, are used, but an embodiment may be adopted in which three or more prediction models are used.

Further, in the embodiment described above, for example, as the hardware structure of the processing unit that executes various processing, such as the acquisition unit 40, the model selection unit 42, the document extraction unit 44, the importance degree derivation unit 46, and the display controller 48, and various processing, such as the acquisition unit 50, the document extraction unit 52, the first evaluation value derivation unit 54, the second evaluation value derivation unit 56, the weight derivation unit 58, the importance degree derivation unit 60, and the display controller 62, the following various processors can be used. As described above, in addition to the CPU that is a general-purpose processor that executes software (program) to function as various processing units, the various processors include a programmable logic device (PLD) that is a processor of which a circuit configuration can be changed after manufacture, such as a field programmable gate array (FPGA), and a dedicated electric circuit that is a processor having a circuit configuration that is designed for exclusive use in order to execute specific processing, such as an application specific integrated circuit (ASIC).

One processing unit may be configured by using one of the various processors or may be configured by using a combination of two or more processors of the same type or different types (for example, a combination of a plurality of FPGAs or a combination of a CPU and an FPGA). In addition, a plurality of the processing units may be configured by using one processor.

A first example of the configuration in which the plurality of processing units are configured by using one processor is an embodiment in which one processor is configured by using a combination of one or more CPUs and the software and this processor functions as the plurality of processing units, as represented by computers, such as a client and a server. A second example thereof is an embodiment of using a processor that realizes the function of the entire system including the plurality of processing units by one integrated circuit (IC) chip, as represented by a system on chip (SoC) or the like. In this way, as the hardware structure, the various processing units are configured by using one or more of the various processors described above.

Further, more specifically, as the hardware structure of the various processors, an electric circuit (circuitry) in which circuit elements, such as semiconductor elements, are combined can be used.

In addition, in each embodiment described above, an aspect is described in which the information processing program 30 or the information processing program 30A is stored (installed) in the storage 22C of the storage unit 22 in advance, but the present disclosure is not limited to this. Each of the information processing program 30 and the information processing program 30A may be provided in a form of being recorded in a recording medium, such as a compact disc read only memory (CD-ROM), a digital versatile disc read only memory (DVD-ROM), and a universal serial bus (USB) memory. Moreover, each of the information processing program 30 and the information processing program 30A may be provided in a form of being downloaded from an external device via a network. That is, an embodiment may be adopted in which the program described in the present embodiment (program product) is distributed from an external computer, in addition to the provision by the recording medium.

In regard to the embodiments described above, the following appendixes will be further disclosed.

Appendix 1

An information processing apparatus comprising: a plurality of models including at least a first model that uses first data as input to carry out a first prediction task and a second model that uses second data as input to carry out a second prediction task different from the first prediction task; and at least one processor, in which the processor is configured to: acquire information data representing information related to a specific target; select a selection model to be used for prediction from among the plurality of models based on the information data; and derive a degree of importance to the specific target for each item included in the information data by using the selection model.

Appendix 2

The information processing apparatus according to appendix 1, in which the processor is configured to: input the information data to the first model to acquire an output result related to the first prediction task; and select any one of the first model or the second model as the selection model based on the output result.

Appendix 3

The information processing apparatus according to appendix 1 or 2, in which the first prediction task is a task for deriving a probability of a first state, the second prediction task is a task related to a second state different from the first state, and the processor is configured to: select the first model as the selection model in a case in which the probability of the first state exceeds a threshold value; and select the second model as the selection model in a case in which the probability of the first state is equal to or lower than the threshold value.

Appendix 4

The information processing apparatus according to any one of appendixes 1 to 3, in which the information data is a document data group including a plurality of document data, the document data group is input to the first model to acquire an output result related to the first prediction task, any one of the first model or the second model is selected as the selection model based on the output result, and the degree of importance to the specific target is derived for each document data included in the document data group by using the selection model.

Appendix 5

The information processing apparatus according to appendix 1, in which the first data is a plurality of first medical information data associated with each of a plurality of patients, and the specific target is a specific patient, the information data is a plurality of second medical information data associated with the specific patient, and the item is each of the plurality of second medical information data.

Appendix 6

The information processing apparatus according to any one of appendixes 1 to 5, in which each of the first prediction task and the second prediction task is a medical-related task.

Appendix 7

The information processing apparatus according to appendix 6, in which the first prediction task is a mortality prediction task, and the second prediction task is a long-term hospitalization prediction task or a complication prediction task.

Appendix 8

An information processing method executed by a processor of an information processing apparatus including a plurality of models including at least a first model that uses first data as input to carry out a first prediction task and a second model that uses second data as input to carry out a second prediction task different from the first prediction task, and at least one processor, the information processing method comprising: acquiring information data representing information related to a specific target; selecting a selection model to be used for prediction from among the plurality of models based on the information data; and deriving a degree of importance to the specific target for each item included in the information data by using the selection model.

Appendix 9

An information processing program causing a processor of an information processing apparatus including a plurality of models including at least a first model that uses first data as input to carry out a first prediction task and a second model that uses second data as input to carry out a second prediction task different from the first prediction task, and at least one processor, to execute a process comprising: acquiring information data representing information related to a specific target; selecting a selection model to be used for prediction from among the plurality of models based on the information data; and deriving a degree of importance to the specific target for each item included in the information data by using the selection model.

Appendix 10

An information processing apparatus comprising: a plurality of models including at least a first model that uses first data as input to carry out a first prediction task and a second model that uses second data as input to carry out a second prediction task different from the first prediction task; and at least one processor, in which the processor is configured to: acquire information data which is a prediction target; use each item data included in the information data as input of the first model to derive a first evaluation value with respect to a first output result related to the first prediction task; use each item data included in the information data as input of the second model to derive a second evaluation value with respect to a second output result related to the second prediction task; use the information data as input of first model to acquire a third output result related to the first prediction task; and perform weighting with respect to the first evaluation value and the second evaluation value by a weight according to the third output result.

Appendix 11

The information processing apparatus according to appendix 10, in which a degree of importance to a specific target is derived for each item by using the weighted first evaluation value and second evaluation value.

Appendix 12

An information processing method executed by a processor of an information processing apparatus including a plurality of models including at least a first model that uses first data as input to carry out a first prediction task and a second model that uses second data as input to carry out a second prediction task different from the first prediction task, and at least one processor, the information processing method comprising: acquiring information data which is a prediction target; using each item data included in the information data as input of the first model to derive a first evaluation value with respect to a first output result related to the first prediction task; using each item data included in the information data as input of the second model to derive a second evaluation value with respect to a second output result related to the second prediction task; using the information data as input of first model to acquire a third output result related to the first prediction task; and performing weighting with respect to the first evaluation value and the second evaluation value by a weight according to the third output result.

Appendix 13

An information processing program causing a processor of an information processing apparatus including a plurality of models including at least a first model that uses first data as input to carry out a first prediction task and a second model that uses second data as input to carry out a second prediction task different from the first prediction task, and at least one processor, to execute a process comprising: acquiring information data which is a prediction target; using each item data included in the information data as input of the first model to derive a first evaluation value with respect to a first output result related to the first prediction task; using each item data included in the information data as input of the second model to derive a second evaluation value with respect to a second output result related to the second prediction task; using the information data as input of first model to acquire a third output result related to the first prediction task; and performing weighting with respect to the first evaluation value and the second evaluation value by a weight according to the third output result.

What is claimed is:

1. An information processing apparatus comprising:
a plurality of models including at least a first model that uses first data as input to carry out a first prediction task and a second model that uses second data as input to carry out a second prediction task different from the first prediction task; and
at least one processor, wherein the processor is configured to:
acquire information data representing information related to a specific target; select a selection model to be used for prediction from among the plurality of models based on the information data; and
derive a degree of importance to the specific target for each item included in the information data by using the selection model.

2. The information processing apparatus according to claim 1, wherein the processor is configured to:
input the information data to the first model to acquire an output result related to the first prediction task; and
select any one of the first model or the second model as the selection model based on the output result.

3. The information processing apparatus according to claim 1, wherein:
the first prediction task is a task for deriving a probability of a first state,
the second prediction task is a task related to a second state different from the first state, and
the processor is configured to:
select the first model as the selection model in a case in which the probability of the first state exceeds a threshold value; and
select the second model as the selection model in a case in which the probability of the first state is equal to or lower than the threshold value.

4. The information processing apparatus according to claim 1, wherein
the information data is a document data group including a plurality of document data,
the document data group is input to the first model to acquire an output result related to the first prediction task,
any one of the first model or the second model is selected as the selection model based on the output result, and
the degree of importance to the specific target is derived for each document data included in the document data group by using the selection model.

5. The information processing apparatus according to claim 1, wherein:
the first data is a plurality of first medical information data associated with each of a plurality of patients, and
the specific target is a specific patient, the information data is a plurality of second medical information data associated with the specific patient, and the item is each of the plurality of second medical information data.

6. The information processing apparatus according to claim 1, wherein each of the first prediction task and the second prediction task is a medical-related task.

7. The information processing apparatus according to claim 6, wherein:
the first prediction task is a mortality prediction task, and
the second prediction task is a long-term hospitalization prediction task or a complication prediction task.

8. An information processing method executed by a processor of an information processing apparatus including a plurality of models including at least a first model that uses first data as input to carry out a first prediction task and a second model that uses second data as input to carry out a second prediction task different from the first prediction task, and at least one processor, the information processing method comprising:
acquiring information data representing information related to a specific target;
selecting a selection model to be used for prediction from among the plurality of models based on the information data; and
deriving a degree of importance to the specific target for each item included in the information data by using the selection model.

9. A non-transitory computer readable medium storing an information processing program causing a processor of an information processing apparatus including a plurality of models including at least a first model that uses first data as input to carry out a first prediction task and a second model that uses second data as input to carry out a second prediction task different from the first prediction task, and at least one processor, to execute a process comprising:
acquiring information data representing information related to a specific target;
selecting a selection model to be used for prediction from among the plurality of models based on the information data; and
deriving a degree of importance to the specific target for each item included in the information data by using the selection model.
